(19) United States
(12) Patent Application Publication
(10) Pub. No.: US 2020/0269003 A1
(43) Pub. Date: Aug. 27, 2020

(54) METHOD FOR OBTAINING RESPIRATORY GAS AT STEADY-STATE BREATHING

(71) Applicant: MediPines Corporation, Anaheim Hills, CA (US)

(72) Inventor: Steve Lee, Anaheim Hills, CA (US)

(21) Appl. No.: 16/810,087

(22) Filed: Mar. 5, 2020

Related U.S. Application Data
(62) Division of application No. 16/280,369, filed on Feb. 20, 2019, now abandoned.
Application No. 16/280,369 is a continuation of application No. 16/131,350, filed on Sep. 14, 2018.
Application No. 16/131,350 is a continuation-in-part of application No. 15/814,902, filed on Nov. 16, 2017.
(60) Provisional application No. 62/430,293, filed on Dec. 5, 2016.

(51) Int. Cl. A61M 16/08; A61B 5/083; A61M 16/00; A61B 5/097

(52) U.S. Cl. CPC A61M 16/085; A61B 5/0833; A61M 16/024; A61B 5/097; A61M 16/0833; A61M 16/049; A61M 2230/432; A61M 2205/3334; A61M 2205/7509; A61M 2205/52; A61M 16/0875

(57) ABSTRACT

The present invention provides a method for obtaining respiratory gas in steady state breathing with minimum contamination of atmospheric air. The breathed air is obtained using a breathing tube having two openings, a breathing opening with an oval shape and an atmospheric opening with a circular shape. The breathed air is passed through a breathing passageway between two openings with laminar flow, and further passed through a sampling hole disposed on the atmospheric opening and coupled to a sampling tube extended halfway into the breathing passageway through the sampling hole.

This application is a divisional application of U.S. patent application Ser. No. 16/280,369, filed Feb. 20, 2019, which is a continuation of U.S. patent application Ser. No. 16/131,350, filed Sep. 14, 2018, which is a continuation-in-part of U.S. patent application Ser. No. 15/814,902, filed Nov. 16, 2017, and claims the benefit of priority of U.S. Provisional Application No. 62/430,293, filed Dec. 5, 2016. These and all other referenced extrinsic materials are incorporated herein by reference in their entirety.

### FIELD OF THE INVENTION

The field of the invention relates to devices with a breathing tube assembly for respiratory gas measurement.

### BACKGROUND

Respiratory gas measurement for steady state breathing is an important assessment to evaluate patient physical condition. The common way to evaluate respiratory gas is to use a mask to completely cover both nose and mouth. Such a mask is described in U.S. patent application US20170197053A1. Because the mask completely covers nose and mouth, patient breathing condition is not normal, patient is not allowed to freely breathe atmospheric air, it is uncomfortable for the patient, and it is not ideal for capturing normal at rest breathing samples requiring steady state breathing.

U.S. Pat. No. 6,779,521 teaches an inhalation device having both ends of the device opened, such that a patient can breathe normally. However, the device is designed to supply oxygen and aerosolized drugs to a patient, not being capable to measure contents presented in patient's breathing.

U.S. Pat. No. 2,882,893 teaches a breathing tube, however, one end of the device is not open to atmospheric air, such that a patient may experience difficulty in breathing. In addition, breathed air must travel a long distance to be analyzed, such that the breathed air will be inevitably mixed with atmospheric air, resulting in inaccurate results.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

There is still need for a device being capable of measuring contents of the respiratory gas at steady state breathing with minimum contamination of atmospheric air.

### SUMMARY OF THE INVENTION

The following description includes information that may be useful in understanding the present invention. It is not an admission that any of the information provided herein is prior art or relevant to the presently claimed invention, or that any publication specifically or implicitly referenced is prior art.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The inventive subject matter provides a breathing tube assembly to measure contents in steady state breathing. Various objects, features, aspects and advantages of the inventive subject matter will become more apparent from the following detailed description of preferred embodiments, along with the accompanying drawing figures in which like numerals represent like components.

A breathing tube assembly comprises a breathing tube having a breathing opening and an atmospheric opening. In some embodiments, the breathing opening is disposed at an angle with respect to the atmospheric opening. More preferably, the breathing opening is disposed at opposite end of the atmospheric opening and two openings are disposed parallel, thereby air flows with no obstruction and can easily exchange breathing air with atmospheric air if it is necessary.

A breathing passageway is fluidly established between the breathing and the atmospheric openings. Furthermore, a sampling opening is disposed between the breathing and the atmospheric openings which allows the monitoring of components presented in the breathed air. Thus, the breathing tube has no port through which air flows besides the breathing, atmospheric and sampling openings.

In a preferred embodiment, a sampling tube is passed through the sampling opening. The distance of the insertion of the sampling tube into the breathing tube is at least 5 mm from the sampling opening. In the most preferred embodiment, the insertion reaches halfway into the breathing tube, maximizing the collection of the breathing air and minimizing the turbulence of the flow at the sampling site. The sampling tube is then fluidly coupled with the transport tube that is extended away from the breathing tube. In some embodiments, the sampling opening is extended vertically to outside and coupled with a transport tube. The coupling is tight enough such that no air leak presents.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

The end of the transport tube extended away from the breathing tube is connected to a monitoring coupling that can contain an electric chip to keep patient ID information and examination results.

In a preferred embodiment, the breathing opening is mated with a mouthpiece. The shape of the mouthpiece has been specifically designed to match a relaxed patient embouchure. The flattened oval opening was designed to limit patient strain as it reflects a natural open-mouthed state. This shape also allows for an air-tight seal, which is important for accurate gas sampling purposes.

The shape of the breathing tube is entirely cylindrical shape or partially non cylindrical. The shape of the non cylindrical portion can be an ovoid or polygonal shape. In some embodiments, the cross-sectional area of the breathing tube is at least 2 cm², keeping air passageway large enough such that a patient can breathe normally (due to laminar flow through the mouthpiece), but also tight enough thereby preventing breathed air contaminated from atmospheric air.

A Polytetrafluorethylene (PTFE) filter or equivalent or a valve is disposed between the breathing opening and the sampling opening. A filter is replaceable or disposable and plays a role to prevent moisture, and capture bacteria and/or virus, preventing monitor electronics from moisture, or bacterial and viral contamination. A valve plays a role to control a flow-direction of the breathing and also can concentrate breathing flow to narrow area, contributing more efficient breathing collection to a sampling tube, providing a more accurate experimental result.

### BRIEF DESCRIPTION OF THE DRAWING

FIG. 1 is a schematic diagram of an embodiment of a breathing tube assembly.

FIG. 1A is a schematic diagram of an atmospheric opening with a reed.

FIG. 1B is a schematic diagram of an atmospheric opening with a screw fitting.

FIG. 2 is a schematic diagram of an embodiment of a breathing tube assembly.

FIG. 3A is a schematic diagram of an embodiment of a breathing tube assembly.

FIG. 3B is a schematic diagram of a sampling tube with a trumpet structure.

FIG. 4 is a schematic diagram of an embodiment of a breathing tube assembly.

FIG. 5 is a schematic diagram of an embodiment of a breathing tube assembly.

FIG. 6A is a schematic diagram of an embodiment of a breathing tube assembly.

FIG. 6B is a cross-sectional view of a breathing tube having a valve.

FIG. 7 is a schematic diagram of an embodiment of a breathing tube assembly.

FIG. 8 is a schematic diagram of an embodiment of a breathing tube assembly.

FIG. 9 is a schematic diagram of an embodiment of a breathing tube assembly.

FIG. 10 is a schematic diagram of an embodiment of a breathing tube assembly.

FIG. 11 is a schematic diagram of an embodiment of a breathing tube assembly.

FIG. 12 is a schematic diagram of an embodiment of a breathing tube assembly.

FIG. 13 is a schematic diagram of an embodiment of a breathing tube assembly.

### DETAILED DESCRIPTION

The present disclosure describes a breathing tube assembly for measuring respiratory gases from a patient. In a preferred embodiment, the breathing tube assembly comprises a breathing tube with two open-ends, breathing opening and atmospheric opening. A patient breathes through the breathing opening and the breathing air can be exchanged to atmospheric air through the atmospheric opening, such that the patient can easily achieve steady-state breathing. In addition, because exchange of air from breathing to atmospheric air does not take a long time, a background level of contents in atmospheric air can be easily monitored. The sampling is obtained by use of the sampling opening where a breathing tube is passed through the sampling opening. The breathing tube is further coupled to a sampling tube and to a transport tube, respectively, allowing to monitor contents of the breathing.

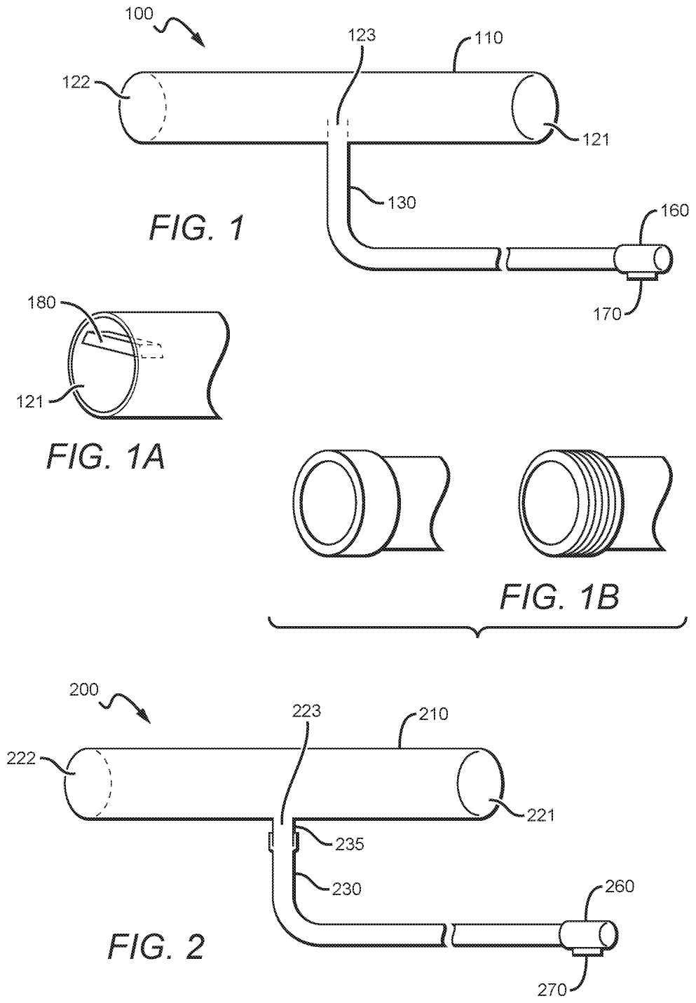

A breathing tube assembly 100 in FIG. 1 generally comprises a breathing tube 110 having two openings, a breathing opening 121 and an atmospheric opening 122 that are disposed parallel each other, conducting breathing passageway fluidly between the breathing and atmospheric openings. Because both ends of the breathing tube are opened, a patient can breathe normally, thus steady-state breathing, in contrast to forced breathing, can be achieved. Besides, the inside of the breathing tube 110 is SPI-A2 surface finished, minimizing resistance to air-flow by maintaining a smooth inner surface, providing smooth air flow.

Furthermore, a sampling opening 123 is disposed between the breathing 121 and atmospheric openings 122 and fluidly coupled with a transport tube 130. The coupling is tight enough to prevent air leak in and out of the tube. The end of the transport tube away from the breathing tube is then coupled fluidly with a monitoring coupling 160. The monitoring coupling contains an electric identification chip 170. The electric chip 170 can store the ID information of the disposable breathing tube assembly in addition to the amount of times the disposable assembly has been used and the time of first use of the assembly. It may also store patient ID and session results. The electric chip can be an encrypted chip.

FIG. 1A shows an embodiment further including a reed 180 configured to be horizontally attached to inside of the breathing opening. If the breathing direction is too high, efficiency of collecting breathing sample gets low, resulting in obtaining an inaccurate result. The reed makes a noise when the breathing direction is too high, such that a patient can re-breathe again to obtain an accurate result.

FIG. 1B shows an embodiment in which the atmospheric opening may have a taper fitting, screw fitting, or specified diameter so that the mouthpiece may be connected tightly to an adapter or other respiratory tubing such that the mouthpiece may be placed in-line with oxygen coming from a ventilator or other respiratory gas device.

In some embodiments, the shape of the breathing tube 110 can be entirely or partially circular, oval or polygonal shape. In a preferred embodiment, the shape of the breathing tube 110 is circular on one end and extended to form a shape of mouthpiece. The mouthpiece is a molded polycarbonate (Makrolon™ 2458) tube that is irregularly shaped and tapered. The shape of the mouthpiece has been specifically designed to encourage and match a relaxed patient embouchure. The flattened oval opening was designed to limit patient strain as it reflects a natural open-mouthed state. This shape also allows for an air-tight seal, which is important for accurate gas sampling purposes. The tight seal prevents an air mixing effect between exhaled air and atmospheric air which would give false measurement data. In commercially viable embodiments, it is advantageous for all materials to have been thoroughly tested for biocompatibility per ISO 10993. Specifically, the materials should pass tests for cytotoxicity, irritation, and sensitization.

FIG. 2 illustrates an embodiment of a breathing tube assembly 200 comprising a breathing tube 210 with a breathing 221 and an atmospheric opening 222 and further comprising a sampling opening 223 which is extended vertically to outside 235. The vertical extension of the sampling opening was further coupled to a transport tube 230. The coupling is tight enough, thereby preventing air in and out of the transport tube. An end of the transport tube 230 away from the breathing tube 210 is connected with a monitoring coupling 260 which contains an electric chip 270.

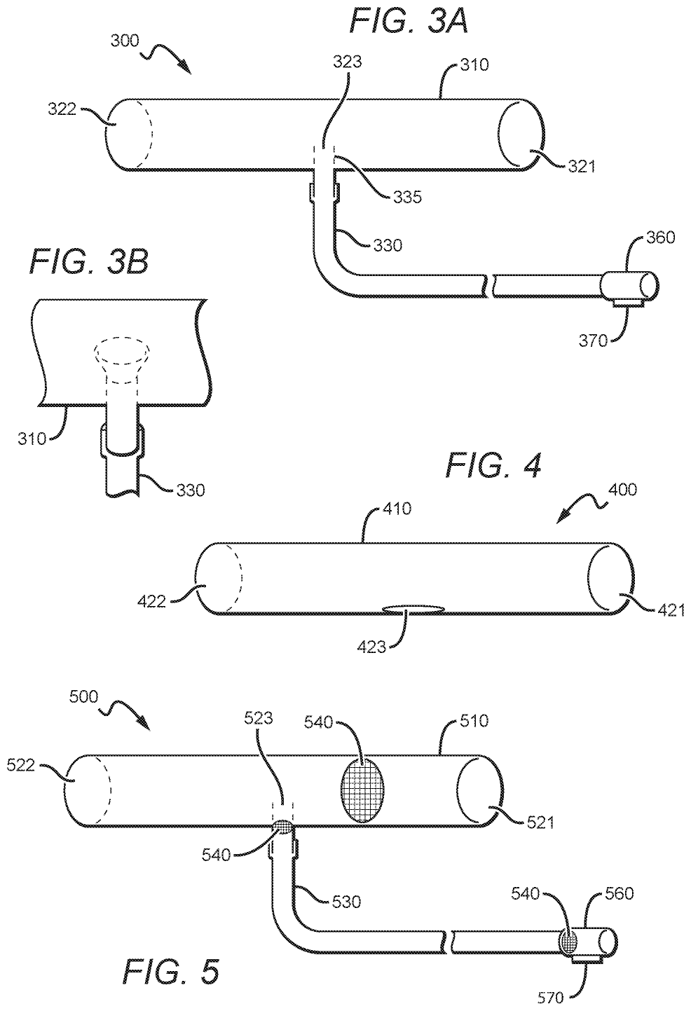

FIG. 3A illustrates an embodiment of a breathing tube assembly 300 comprising a breathing tube 310 with a breathing 321 and an atmospheric opening 322 and further comprising a sampling tube 335 that is inserted through the sampling opening 323. In a preferred embodiment, the sampling tube is extended into about the halfway inside of the breathing tube 310. Thus, air drawn into the sampling tube comes mostly from breathing air but not from an atmospheric air. In some embodiments, the end of the sampling tube inside of the breathing tube can be enlarged like a trumpet (FIG. 3B), such that sample collection achieves more efficiently. An end of the sampling tube away from the breathing tube is coupled with the transport tube 330, and an end of the transport tube away from the breathing tube is further connected to a monitoring coupling 360. The monitoring coupling can contain an electrical chip 370.

FIG. 4 illustrates a basic embodiment 400 comprising a breathing tube 410 with three openings, a breathing 421, an atmospheric 422 and a sampling 423 openings. Indeed, the breathing tube has no port through which air flows besides the breathing, atmospheric and sampling openings. An external tube can be inserted into the sampling opening 423, allowing to monitor contents of the breathing air.

FIG. 5 illustrates an embodiment of a breathing tube 500 assembly comprising a breathing tube 510 that has a breathing 521 and an atmospheric opening 522. A transport tube 530 is passed through a sampling opening 523 and an end of the transport tube away from the breathing tube is further connected to a monitoring coupling 560 that can contain an electrical chip 570. In a preferred embodiment, a filter 540 is disposed in air pathway between the breathing opening 521 and the atmospheric opening 522, and/or inside of the sampling opening 523 transport tube, and/or inside of the monitoring coupling 560. The filter protects a monitoring machine from moisture and/or bacterial/virus contamination and can be replaceable or disposable. The pore size of the filter can be between 0.22 μm and 0.45 μm.

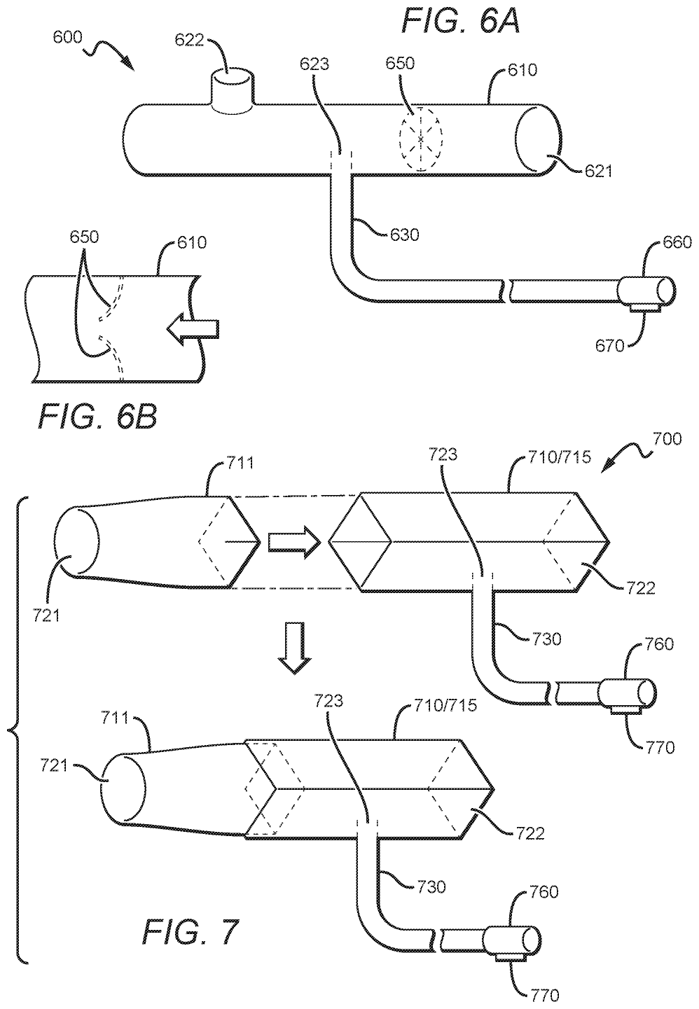

FIG. 6A illustrates an embodiment of a breathing tube assembly 600 comprising a breathing tube 610 that has a breathing 621 and an atmospheric opening 622 where the breathing opening 621 is angled with respect to the atmospheric opening 622. A valve 650 is disposed inside of the breathing tube 610. In an embodiment shown in FIG. 6B, the valve can be opened when breathing flow comes in. The transport tube 630 is passed through the sampling opening 623 and a connection between the transport tube 630 and the sampling opening 623 is tight enough to achieve no air leak in and out of the tube. An end of the transport tube away from the breathing tube is further connected to a monitoring coupling 660 that can contain the electrical chip 670.

FIG. 7 illustrates an embodiment of a breathing tube assembly 700 comprising a breathing tube 710 physically separated to two parts a first 711 and second 710 breathing tubes. Both ends of the first and second tubes are opened. The first breathing tube 711 has a breathing opening 721, and the second breathing tube 710 has an atmospheric opening 722 and a sampling opening 723. An opposite opening of the breathing opening 721 is sized and dimensioned to be coupled to an opposite opening of the atmospheric opening 722. The coupling is tight enough, such that no air leak is observed in and out of the breathing tubes. A transport tube 730 is passed through the sampling opening 723 and an end of the transport tube away from the breathing tube is further connected to a monitoring coupling 760 that can contain an electrical chip 770.

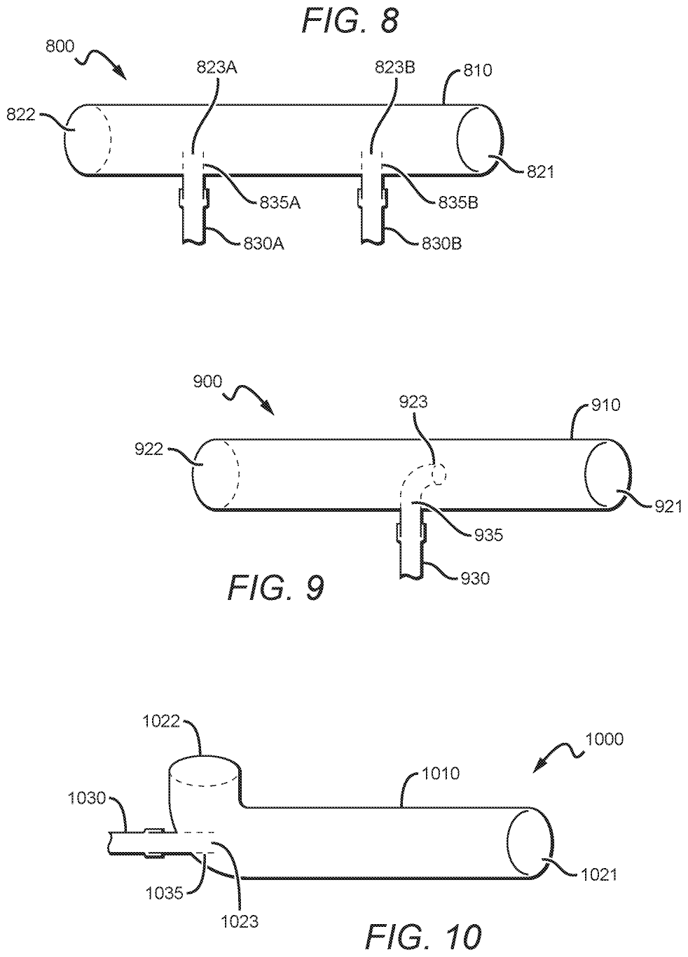

FIG. 8 illustrates an embodiment of a breathing tube assembly 800 comprising a breathing tube 810 with a breathing opening 821, an atmospheric opening 822, and a plurality of sampling openings 823A, 823B, each coupled with a sampling tube 835A, 835B, and a transport tube 830A and 830B, respectively. It is contemplated that the various sampling openings could be distributed along the breathing tube 810 in any desired locations, and there could be more than two such openings.

Additionally, as with the other figures, the various sampling tubes could extend into the lumen of the breathing tube at any desired distances, and the various sampling tubes could have any desired cross-sectional sizes and shapes, and could terminate horizontally as shown, or be angled, bent or tilted in some manner.

FIG. 9 illustrates an embodiment of a breathing tube assembly 900 comprising a breathing tube 910 having a breathing opening 921 and an atmospheric opening 922 and further comprising a sample opening 923. The sample opening 923 opens to a bent sampling tube 935, which is removably coupled with a transport tube 930. Alternatively, the sampling tube 935 could be bent towards the atmospheric opening 922 or directed at some oblique angle with respect to the breathing and atmospheric openings 921, 922, respectively.

FIG. 10 illustrates an embodiment of a breathing tube assembly 1000 comprising a breathing tube 1010 having a bend fluidly disposed between a breathing opening 1021 and an atmospheric opening 1022. In a preferred embodiment, a sampling opening 1023 is disposed near the bend, with a sampling tube 1035 substantially parallel to a long axis of the main portion of the breathing tube 1010. In related embodiments, the sampling opening 1023 and sampling tube 1035 could be angled otherwise than that shown, and could be positioned elsewhere along the breathing tube 1010. Sampling tube 1035 is removably coupled to transport tube 1030.

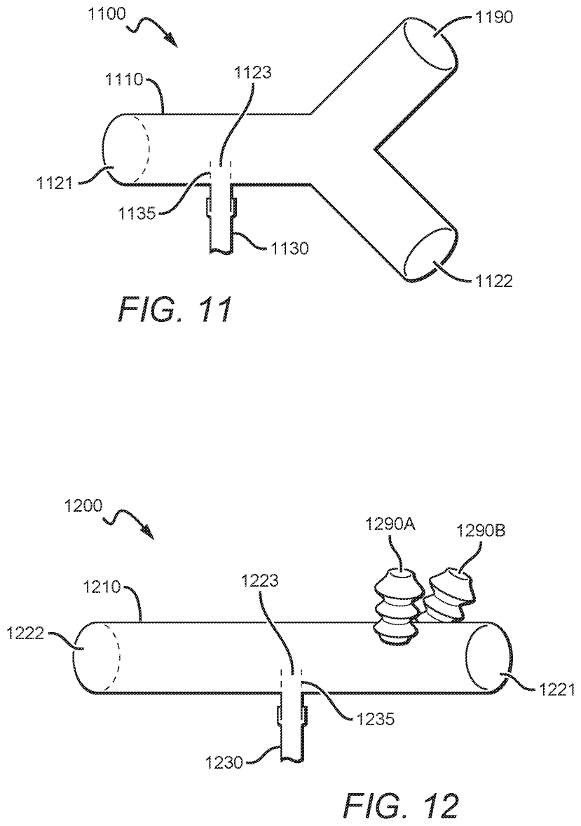

FIG. 11 illustrates an embodiment of a breathing tube assembly 1100 comprising a breathing tube 1110 having a breathing opening 1121, an atmospheric opening 1122, and supplemental oxygen passageway opening 1190. A sampling opening 1123 leads to a sampling tube 1135, which in turn is removably coupled to transport tube 1130.

FIG. 12 shows an embodiment of a breathing tube assembly 1200. In this embodiment, a breathing tube 1210 has a breathing opening 1221 and an atmospheric opening 1222, and further includes two nasal cannula consists of two flexible tubes 1290A/1290B, sized and dimensioned to fit into the two openings of a patient's nose so that the patient may breathe through the nose and mouth. A sampling opening 1223 leads to a sampling tube 1235 and is removably coupled with a transport tube 1230.

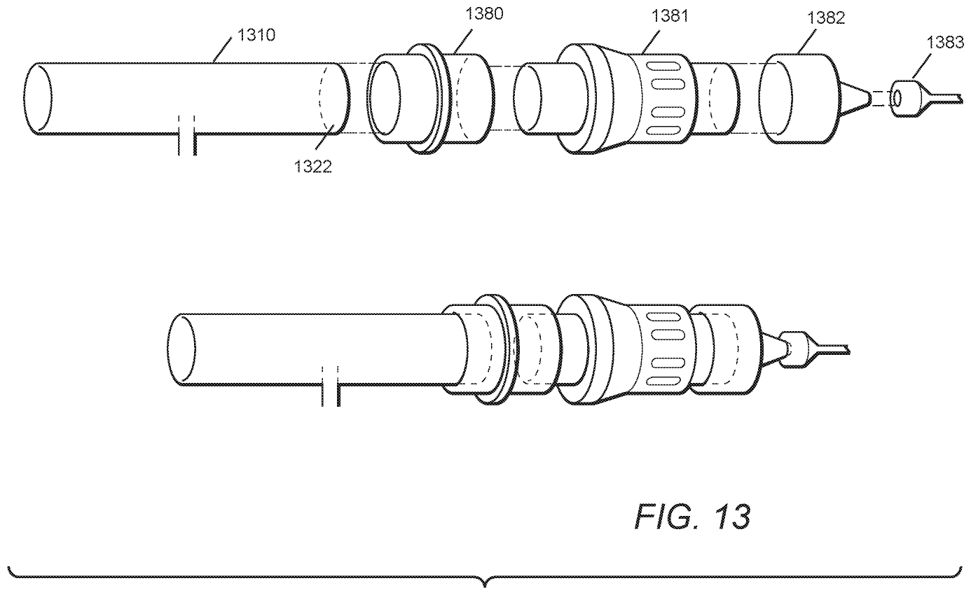

FIG. 13 shows an embodiment of a breathing tube assembly. In this embodiment, several adapters (1380-1383) may be attached to the atmospheric opening 1322 of the tube. The adapters allow the breathing tube 1310 assembly to be in-line with a ventilator or with other supplied respiratory gases. One of the adapters 1381 may have small vents to facilitate patient breathing gas outflow to atmosphere.

It should be apparent to those skilled in the art that many more modifications besides those already described are possible without departing from the inventive concepts herein, he inventive subject matter, therefore, is not to be restricted except in the scope of the appended claims. Moreover, in interpreting both specification and the claims, all terms should be interpreted in the broadest possible manner consistent with the context. IN particular, the terms “comprises” and “comprising” should be interpreted as referring to elements, components, or steps in a non-exclusive manner, indicating that the reference elements, components, or steps may be present, or utilized, or combined with other elements, components, or steps that are not expressly referenced, Where the specification claims refers to at least on one of something selected from the groups consisting of A, B, C, . . . and N, the next should be interpreted as requiring only one element from the group, not A plus N, or B plus N, etc.

What is claimed is:

1. A method of sampling steady state breathing air of a person, comprising:
providing a breathing tube having an oval breathing opening, a circular atmospheric opening, and a breathing passageway between the breathing and atmospheric openings, wherein the breathing passageway is tapered from the breathing opening to the atmospheric opening,
positioning the person's mouth about the oval breathing opening, and having the person breathe in and out through the breathing tube in a steady state manner, such that breaths of the person pass with laminar flow through the breathing passageway; and
using a sampling tube coupling an opening disposed within the breathing passageway to sample air from a breath passing through the breathing passageway.

2. The method of claim 1, wherein the sampling tube is positioned within an elongated, cylindrical portion of the breathing tube.

3. The method of claim 1, further comprising passing the air through a filter disposed within the passageway.

4. The method of claim 1, further comprising passing the air through a valve disposed within the passageway.

5. The method of claim 1, further comprising providing the breathing passageway with a normal cross-sectional airflow area of at least 1.0 cm².

6. The method of claim 1, further comprising coupling the sampling tube to a transport tube.

7. The method of claim 6, further comprising coupling a monitor at an end of the transport tube.

8. The method of claim 1, further comprising using an identification chip coupled to the breathing tube to ascertain a number of times that the breathing tube has been used.

9. The method of claim 1, further comprising using an identification chip coupled to the breathing tube to store patient ID and session results.

10. The method of claim 1, further comprising placing the breathing tube in-line with a ventilator or other respiratory device delivering gas to a patient.
